(19) United States
(12) Patent Application Publication
(10) Pub. No.: US 2023/0264038 A1
(43) Pub. Date: Aug. 24, 2023

(54) Method for Microwave Treatment and System for Same

(71) Applicant: Hypersound Medical, Inc., Scottsdale, AZ (US)

(72) Inventor: Bruce C. Towe, Phoenix, AZ (US)

(73) Assignee: Hypersound Medical, Inc., Scottsdale, AZ (US)

(21) Appl. No.: 18/112,954

(22) Filed: Feb. 22, 2023

Related U.S. Application Data
(60) Provisional application No. 63/313,275, filed on Feb. 24, 2022.

(51) Int. Cl. A61N 5/04

(52) U.S. Cl. CPC A61N 5/04

(57) ABSTRACT

A method for treating a patient that includes directing first microwave energy towards excitable tissue in a patient and second microwave energy towards the excitable tissue at an angle from the first microwave energy. The first electric field from the first microwave energy overlaps with the second electric field from the second microwave energy at the excitable tissue so as to alter a physiological function of the excitable tissue. Third microwave energy can optionally be directed towards the excitable tissue for overlapping with the first microwave energy and the second microwave energy at the excitable tissue.

### CROSS REFERENCE TO RELATED APPLICATION

This application claims priority to U.S. provisional patent application Ser. No. 63/313,275 filed Feb. 24, 2022, the entire content of which is incorporated herein by this reference.

### FIELD OF INVENTION

This invention relates generally to microwave neuromodulation, and more particularly to microwave neuromodulation for treating pains and disorders in a human.

### BACKGROUND OF THE INVENTION

Direct application of relatively low frequency pulsed electrical currents has been used for the medical treatment of pain and neurological disorders in humans.

Most all vital organs of the human body are under control of the central nervous system (CNS), which generally includes the brain and spinal cord. The control of nerve, muscle, and organs by artificial manipulation of their bioelectrical nature by applied electrical currents has been known as a route to their functional control. Such control is known to produce relief of pain, restored organ function, aspects of healing, regeneration, and homeostasis. Methods of applying electricity to the human body are currently used for the therapeutic treatment of epilepsy, stroke, mood disorders, cardiac arrhythmia's, Parkinson's disease, neuralgia and peripheral vascular disease.

Current implant neuromodulation devices for pain relief and certain neurologic disorders generally involve relatively bulky and pacemaker-like powered pulse generators, whose silicone catheter-type electrodes must be surgically routed in the body to their therapeutic target site. They are then set to deliver low level electrical currents to alleviate symptoms. This approach is undesirable in that it requires surgery and its attendant costs and hazards of infection, dislodgement of electrodes, battery issues, mechanical failures, and problems with patient magnetic resonance imaging (MRI).

Transcutaneous electronic nerve stimulators (TENS units) are often prescribed for pain relief by applying an electrical current through the skin to subsurface nerves. Current neurostimulation for the relief of pain is generally theorized to work by way of the Gate Theory of pain, wherein applied electrical currents cause preferential stimulation of the large A fiber subtypes in a nerve bundle. A fibers are the fast-reflexive motor and sensory fibers in a nerve bundle. By human neural wiring, the brain preferably pays more attention when A fibers are activated and less attention to the less electrically sensitive and smaller C fibers in the same nerve bundle, which carry deep pain. Current theory is that gentle stimulation of A fibers at a low level is effective in reducing pain because the brain is occupied by a buzzing sensation carried by the A fibers, to which the brain accommodates, and pays less attention to deep and chronic pain signals from C fibers. These technologies have drawbacks in that they can create unpleasant shock sensations since current intended for A fibers often must be relatively high to overcome losses through the insulating quality of the skin and so actuate shallow C fibers as well. In addition, these devices are often uncomfortable in that they require skin electrodes and the electrical pulses applied can produce a prickly discomfort or even pain at the electrodes.

A neuromodulation alternative to TENS is spinal cord stimulation (SCS), which places surgically implanted electrodes directly onto the spinal cord. This approach is intended to block pain at a higher organizational level at the spinal cord signal to the brain and does not stimulate pain receptors in the skin.

There are many problems with the use of electrical stimulation of neural tissues for medical therapeutics. For example, the inability of electrical currents to be finely tuned to stimulate only the desired nerve fibers within a close packed bundle. Currently, the whole nerve is stimulated in order to reach the fibers that are effective. However this undesirably stimulates others in the same bundle and so producing side effects. Further, electrical stimulating currents can locally damage tissues, particularly at higher current level settings, through local heating and pH changes. The use of silicone catheters and bulky implanted batteries can be uncomfortable to the patient. Surgically implanted electrodes and battery powered pulse generators involve risks of surgery and include high costs, potential for infection, problems with reliability, mechanical and electrical issues with the placement of pulse generators, off-target effects, lack of precise control, and interference with common medical imaging technologies.

Microwave energy at elevated power levels has long been known to produce heating of biological tissues and has been used in medical diathermy to relieve muscle aches and pain. It is also used in cancer therapy for purposes of destroying tumors by locally overheating tissues by one or more body surface mounted patch antennae designed so as to localize heating to just the tumor itself. Microwave energy applied to nervous tissue of a human at frequencies, for example in the roughly the 1-10 GHz range, commonly used in microwave communications, for example by cell phones, is known to show no stimulatory effects on nerves.

Microwave energy at higher frequencies of 30 GHz and above are reported by several investigators to have effects on neural tissues and particularly effects on nerve membrane potentials leading to their greater excitability. However, at these high frequencies the penetration depth of microwave energy in biological tissues is limited to a few millimeters at most and thus not useful as a means of medical neuromodulation. Employing relatively higher power levels in an attempt to reach more deeply in tissues causes problems of overheating of near tissues.

It is known that microwave energy can have nonthermal effects on biology. However at frequencies below about 30 GHz microwaves are not known to the art as a method of direct neurostimulation.

In view of the foregoing, there is a need to find a better way of stimulating nerve, tissues, and organs that does not have the foregoing problems.

### BRIEF DESCRIPTION OF THE DRAWINGS

FIG. 1 is a schematic drawing of an embodiment of a microwave treatment system of the invention, and shows three microwave generators and three microwave wave emitting devices producing a bioactive zone within a larger biological tissue of a patient.

FIG. 2a is a graph of a sample combination of three microwave electromagnetic fields at 120 degree phase relationship emitted each from one of three wave emitting devices, for example from the microwave treatment system of FIG. 1.

FIG. 2b is a graph of sample resultant induced forces within a solid medium, for example from the electromagnetic fields of FIG. 2a.

FIG. 3a is a graph, on a different time scale from the graph in FIG. 2a, of pedestal wave of high frequency forces, for example from the electromagnetic fields of FIG. 2a.

FIG. 3b is a graph of the result when there is an amplitude modulation impressed on the generating electric field forces, for example from the electromagnetic fields of FIG. 2a.

FIG. 4 is a drawing of the components of an embodiment of a microwave treatment system, for example the microwave treatment system of FIG. 1.

FIG. 5 is a computer generated output of a computational mode of three 1 GHz frequency hypersound vibration waves rotated 120 degrees to each other and crossing in the center to form a triangular zone of interference that produces a characteristic moiré pattern of interference and sub-micron diameter cells of force, for example from the microwave treatment system of FIG. 1.

FIG. 6 illustrates an embodiment of a microwave applicator employing three equi-spaced wave emitting devices and their projected electric fields that cross in a central area.

FIG. 7 illustrates an embodiment of an application of a microwave treatment system of the invention to the lower back a patient, where the wave emitting devices of the system project three electric fields through targeted tissues of the back to stimulate the spinal cord.

FIG. 8 illustrates a sample pulsed and oscillating signal waveform used to modulate the three microwave source amplitudes.

FIG. 9 illustrates an application of microwave energy from three microwave wave emitting devices of the system of the invention to a heart region through the ribcage, seen in cross section, and directed at the atrioventricular (A-V) node.

FIG. 10 illustrates an application of microwave energy from three microwave wave emitting devices of the system of the invention to a heart region through the ribcage and directed at a myocardial infarction.

### DETAILED DESCRIPTION OF THE INVENTION

The method and system of the invention can optionally be used for treating tissue of a patient. The system of the invention can optionally be referred to as a bioactive wave device or system. The tissue can optionally be deep tissue, separated from or spaced below the skin of the patient by intervening tissue. The targeted tissue can optionally be referred to as a biological target. The tissue can optionally be excitable tissue of any type. The excitable tissue can include a nerve, a spinal nerve, a vagus nerve, a trigeminal nerve, an occipital nerve, a sacral nerve, a peripheral nerve, a sensory organ, an organ for sensing pressure, an organ for sensing temperature, an organ for sensing proprioception, an organ having sensitivity to pressure, a neural components of the eye, a neural component of the ear, an excitable neural ganglia, a brain cortex, white matter, gray matter, a tissue of the frontal lobe, a tissue of the parietal lobe, a tissue of the occipital lobe, a tissue of the temporal lobe, a brain organ, a hippocampus, a thalamic nuclei, an endocrine gland, muscle, smooth muscle, skeletal muscle, cardiac muscle, skin cells, hair follicles, teeth, gums, vasculature, a region of the heart, a sinoatrial node, an atrioventricular node, a myocardium or any combination of the foregoing.

The treatment can be of any suitable type, optionally including altering a physiological function of the targeted tissue. The alteration of the physiological function can optionally include an initiation of an action event, a neural subthreshold bioelectric change, a change in spontaneous rate of cell bioelectrical firing, an alteration of muscle contraction or any combination of the foregoing. The targeted tissue can optionally include a nerve bundle, with the treatment providing a desired neurostimulation effect from the nerve bundle. The targeted tissue can optionally include a vertebral region of a back of the patient and the surrounding spinal ganglia regions, with the treatment providing a neurostimulation effect that results in a reduction of pain in the vertebral region. The nerve bundle can optionally include an A class of fibers, a B class of fibers and a C class of fibers, with the treatment providing a desired neurostimulation effect to less than all of the class of fibers of the nerve bundle or to only one class of fibers. The targeted tissue can optionally include a region of the heart, with the treatment capturing an irregular heart rhythm and thus acting liking a pacemaker. The targeted tissue can optionally be an area of an infarction in a heart, with the treatment causing a physiologic change at a cellular level that increases the functionality of cardiac cells, the physiologic change optionally including an increase of myocardial blood flow through vasodilation, an arrest of arrhythmias, an arrest of aberrant beats or any combination of the foregoing. The targeted tissue can optionally be an area of an infarction in a heart, with the treatment leading to a reduction of ischemic tissues that might otherwise undergo necrosis and optionally including increased bioelectrical activity, improved tissue metabolism, increased blood flow or any combination of the foregoing.

The method and system of the invention can optionally include directing microwave energy with electric fields from a plurality of directions at the targeted tissue so that the electric fields overlap, intersect or cross at the targeted tissue to treat the targeted tissue. The directed microwave energy can optionally be referred to as a microwave beam. The electric fields can optionally be referred to as vector electric fields. The microwave energy can be directed from any plurality of directions at an angle to each other, including two directions, three directions, four directions and up to n directions. The directions of microwave energy can optionally be disposed about the targeted tissue, for example symmetrically disposed about the targeted tissue so that the respective electric fields symmetrically overlap at the targeted tissue. For example, microwave energy can be provided from three directions that are symmetrically disposed at separation angles of approximately 60 degrees about the targeted tissue so that the three beams of microwave energy, and respective electric fields or vector electric fields, symmetrically overlap at the targeted tissue. Slight deviations in symmetrical or other spacing of microwave beams, for example +/−10% deviations, can be acceptable and encompassed by the term approximately. The directions of microwave energy can optionally be disposed in a plane that contains the targeted tissue.

The method and system include at least one microwave source of any suitable type for providing the plurality of microwave energy beams directed at the targeted tissue. A controller is coupled to the at least one microwave source and optionally includes a processor, storage and software, firmware, hardware or any combination of the foregoing for operating the at least one microwave source. A single microwave source can optionally be provided that includes a plurality of wave emitters of any suitable type for respectively providing a plurality of microwave energy beams, a separate microwave source and wave emitter can optionally be provided for each microwave energy beam or a combination of the foregoing can be provided. A wave emitter can be provided for producing each microwave beam and can be of any suitable type, including a wave emitting device, an emitting field element, a microwave field generator, a microwave dipolar antenna, a microwave patch antenna, a microwave waveguide, an antenna employing dielectric matching layers to the body surface of the patient, an exposed coaxial cable, a skin contacting electrode, a wholly tissue-implanted antenna-electrode system or any combination of the foregoing. Any of all of the wave emitters can optionally be spaced away from the skin of the patient, for example for placement outside the clothes of the patient, in a wearable device or both.

The operating parameters of the at least one microwave source, and each microwave energy beam and corresponding electric field, can be configured, controlled or determined by any suitable combination of operating parameters, including frequency, power, phase, pulse duration, repetition rate, pulse amplitude modulation or any combination of the foregoing. Each microwave energy beam can optionally be related by phase or have the same phase. Each microwave energy beam can optionally be of single frequency having controllable amplitude and phase. Each microwave energy beam can optionally have a frequency ranging from 300 MHz to 300 GHz. Each microwave energy beam can optionally have a frequency ranging from 0.9 to 6.0 GHz. Each microwave energy beam can optionally be tuned to a different frequency. Each microwave energy beam can optionally be tuned to a microwave carrier frequency and a pulse modulation frequency, for example in a neuromodulation treatment. Each microwave energy beam can optionally be appropriately tunes to achieve a desired neurostimulation effect, for example to only one of the class of fibers in a nerve bundle. The controller can be configured, for example by software, firmware, hardware or any combination of the foregoing, to control the desired characteristics of each microwave energy beam, including any of the characteristics and parameters disclosed herein.

The method and system of the invention can optionally be configured to project three said electric field vectors from sites at the corners of an equilateral triangle inward towards a central enclosed zone containing the targeted tissue, which can be referred to as the biological target, wherein the electric field vectors are oriented to produce crossing of the electric fields within the space of the biological target. The method and system of the invention can optionally be configured to provide a plurality of functionally associated triads of microwave energy beams related by phase, wherein each triad is positioned with other functionally associated triads in a three dimensional configuration. Each triad can optionally be energized in sequence so as to provide a greater diversity of the electric field vector directions at the biological target, thereby producing greater effectiveness of the treatment of the biological target. The at least one microwave source can optionally be configured to tune each triad of microwave energy beams at a different carrier frequency, for example to provide physiologic change in two or more cell types or alternately two or more different cellular functions of the same cell type at the same time. The at least one microwave source can optionally be configured to provide that the pulse amplitude modulation includes two or more frequencies so as to induce physiologic change in two or more cell types or alternately two or more different cellular functions of the same cell type at the same time.

Under the method and system of the invention, neural and excitable tissues targeted by multiple intersecting microwave vector fields can experience unexpected neurostimulatory effects, for example from the generation of hypersonic frequency waves by microwave electrostriction and the sensitivity of cellular ion channels to physical distortion by hypersonic vibration.

Electrostriction is a quadratic, nonlinear electromechanical interaction. Treating a dielectric membrane as part of a parallel-plate capacitor, the pressure P on a dielectric due to an electric field applied across it can be expressed as:

P=εᵣε₀E² eq. 2

where εᵣ is the dielectric constant of the membrane, co is the permittivity of free space, and E is the electric field. Electrostrictive pressures are proportional to the material dielectric constant εr which in most matter is on the order of εr=1 to 3. The higher this number, the greater the induced force amplitude by eq. 2.

Biological cell membranes however have very high dielectric constants, as much as εr=100 and so are 50× times that of common materials in the environment. Thus at a given electric field power, electrostrictive pressure at cell membranes, for example, are much higher than might be otherwise expected from experience with conventional dielectrics. The resultant pressure has both a steady-state average value as well as a monophasic oscillating value when driven by oscillating electromagnetic waves.

Electrostrictive vibration of living tissues is the same frequency as the frequency range, for example GHz range, of the inducing microwave field and so the vibrations are in the range of what is known as hypersound. Hypersound vibrations have very short wavelengths, for example tens of nanometers, and so are effective in coupling to cellular scales of physiology. Thus cellular neurostimulation effects of multiple microwave sources overlapping from different vector directions are considered to be a result of interfering electrostrictive forces that create complex patterns of interference. Criss crossing hypersonic waves are believed to create shearing mechanical forces at cellular structures, including cellular ion channel operation, resulting in inflows of sodium ion and thus neurostimulation. In addition, the pressure and electric field are vectors so a mathematical dot product exists, meaning that the pressure is in the direction of the electric field. Because of the square law effect the force frequency is twice that of the frequency of an applied oscillating electric field.

Microwaves in the GHz frequency range applied to living things produce, by physical theory, oscillatory forces on dielectric cell membranes that produce in-situ vibrations by way of effects of electrostriction. Vibration induced in matter at GHz frequencies is known as hypersound to physicists, but are currently not measurable since there are no instruments that can follow such fast vibrations. However, hypersound characteristics can be predicted since hypersound is known to follow the usual rules of acoustic wave phenomena.

Microwave energy applied to dielectrics at GHz frequencies produce wavelengths of hypersonic vibration that follow the relationship:

λ=c/f eq. 3

where c is the speed of sound (usually taken to be about 1500 m/sec in tissue), f is the frequency, and λ is the acoustic wavelength. Induced electrostrictive vibrations in dielectric matter at microwave frequencies, taking into account the frequency doubling effect of electrostriction, thus have wavelengths on the order of several hundred nanometers and comparable to the sizes of subcellular dimensions. The high frequencies of these waves classifies them as hypersonic, which distinguishes them from waves in the lower ultrasonic region used in current medical applications.

Thus, the microwave frequency applied to matter determines the dimensions where forces are experienced. The pressure gradient is the operable quantity at the cellular nanoscale and only becomes significant when vibrations have such short wavelengths. This presents a unique capability for applications of microwave induced forces on cellular-scale biology. In this regard, the common use of ultrasonic forces in the medical range of MHz cannot produce much force differential shear on cellular or subcellular structures. This is because the wavelengths of such ultrasonic forces are millimeter-order and micrometer-order sized cells that just ride along with such wave like a cork on the ocean and experience little differential force. In contrast, hypersound produces compression and rarefactions at the scale of cellular structures and so are operative at this level.

Under the method and system of the invention, it is possible to evoke a range of vibrations upwelling within tissue that are active at subcellular scales because of their short wavelength. The forces are specifically localized to membranes because of their dielectric qualities, and at the same time produce acoustical vibrations at these locales in the ranges of sonics and ultrasonics. Physical laws of acoustics apply in terms of generation, propagation, and resonances. Acoustic resonances are a particularly powerful method of transferring and magnifying mechanical wave energy by integration of energy over time. Mechanical resonances are well known to be a strong function structure and composition.

Relevant to the method and system of the invention is the average positive value of force that arises due to the squared term in equation 2. In addition to a hypersound vibration, there is superimposed additional average positive force over the time the microwave pulse is on. This might be likened to a sustained push or pinching on tissue as if the tissue were gripped by an unseen internal process rather than experiencing disturbing forces from outside. The force can appear within biological tissues along the microwave penetration path.

This monophasic push on cells along the microwave penetration path can be further constrained to a specific locale in 3D space by the overlapping conjunction of multiple microwave beams. In a sense, this allows a more directed and focusing of effect to the targets such as specific nerves or organs inside mammals. In addition, the use of a plurality of microwave beams, for example three, in a crossed and phased geometry produces a hypersound interference effect where, instead of long ripples of hypersound waves parallel to an electromagnetic wave direction, there are formed fixed nanoscale triangular zones of pressure and rarefaction.

These closed regions of oscillating high frequency forces can capture cellular subcomponents in zones of higher force effect by way of shearing that occurs between zones of compression. The shear forces that arise on membranes is an aspect employed by the method and system of the invention and, by a theory interactive with cellular membrane function, are able to modulate cellular events, for example result in an alteration in cell physiologic function and bioelectrical action events.

Under the method and system of this invention, two or more microwave wave emitting devices or sources may be employed in a manner that there is an angle between them, for example so they do not appear as a single source. Three microwave beams can be utilized, for example in a triangular arrangement around a target forming nominally 120 degree crossing angles. In addition, mutual 120 degree microwave phase relationships between the three crossing fields may maximize evoked hypersound wave shearing effects.

Since higher frequency microwave energies produce finer wavelengths of vibration, microwave frequency can be a defining variable in effective neuromodulation because waves tend to couple to cellular scale structures of comparable size. Thus, the physiologic effects of microwave energies can be targeted to cellular structures by the choice of the microwave carrier frequency.

When displayed in a computer 2D model of hypersound pressure versus position during a pulse, the zone of microwave crossing shows a steady central pressure pedestal with a superimposed high frequency hypersound induced grid of pressure points. Pedestal wave vibration effects, for example, can be demonstrated by placing a test dielectric at the conjunction of microwave beams as described, and optionally modulating the microwave intensity at kHz to MHz frequencies. As the modulation of the microwave is swept over a range, the test dielectric will typically show vibrational resonant responses at highly specific frequencies. These responses can depend on test dielectric size, shape, elasticity, composition, and other acoustic qualities.

Under the method and system of the invention, an additional vibrational force can be produced by oscillatory modulation of the pedestal amplitude at some frequency which is generally in the kHz-MHz range. Cellular membrane structures can thereby feel an additional pulsatile vibration at a secondary lower frequency, which augments the hypersound grid of pressure points. There can thus be a combinational vibrational effect of two different applied frequencies, one by the chosen microwave carrier and another by its modulation frequency, that currently cannot be achieved in any other way.

The method and system of the invention can produce vibrations that behave in tissues according to laws of lower frequency acoustics. Such forces can be generated within tissue over a wide range from kHz to high MHz. For example, medical range ultrasonic forces in the multi-megahertz region can be created without the requirement for a surface-emitting transducer.

The method and system of the invention can create vibratory forces within tissues both over a very wide frequency range and at very fundamental levels of cellular nanoscale organization. It is possible by such method and system to vibrate into altered and generally greater activity those structures within cells that are resonant to a chosen frequency and not other structures in dissonance. The ability of such method and system to be selective in imparting activation to specific cell membrane structures by resonance and not to others offers utility and advantage over current techniques of stimulation. In this regard, such method and system can achieve selective effects on living cells not possible with current electrical stimulation.

A mechanism of action is suggested in the following paragraphs by which electromagnetic microwaves induce physiological changes originating at cellular scales, and involves induced vibration at the cell membrane levels. The effects of vibration are a result of flexing and distortion of the stereochemical nature of cell membranes and consequently their ion channel functionality. Increased diffusion effects by vibration create a more mobile supply of molecules to active sites for living processes to occur.

Under the method and system of the invention, microwave induced electrostrictive forces are an agent whereby cellular-scale forces are generated and which can be easily tuned to resonances or preferred frequencies of vibration of different membrane molecular structures. These membrane molecular structures vary in structure and function, such as cellular plasma membrane, endoplasmic reticulum, golgi apparatus, and nuclear membranes leading to enhanced metabolic processes. Such method and system use microwave frequency and modulation as a method for selectively stimulating cellular physiologic events by tuning the microwave energy to their natural frequency.

The method and system of the invention are advantageous over known electrical stimulation techniques in that biowave actions are acoustic in nature and so follow these laws which are much different and useful in this case than the laws of electromagnetism. As a result, there is an ability to be selective in terms of physiologic susceptibilities to different frequencies of induced bioactive vibration.

Microwave power levels needed to achieve biological effects can be relatively high peak pulse power, when compared to most environmental transmitting sources, but of a moderate and biologically safe average power. The microwave electromagnetic field level is sufficient to overcome absorption by overlying tissue and bone structures to achieve penetration to a target. Depending on the application, the microwave power levels can vary over a wide power range, taking into account limitations by safety considerations of microwave heating. Electrostriction is proportional to the square of EM intensity, so relatively moderate increases in microwave power can cause large changes in effect.

Any suitable parameters for operating a microwave energy source can be utilized, including frequency, power, phase, pulse duration, repetition rate, and pulse amplitude modulation. Such parameters of the applied microwave energy source interact to produce different effectiveness and specificity in physiologic effects. The microwave carrier frequency to a large extent can define the penetration depth of the electromagnetic waves and thus can optionally be utilized to select tissue targets nearer to the skin rather than far.

Bioactive wave effects of the method and system of the invention occur in a localized manner, which can be some mid-point between the microwave antennas other wave emitting devices. The three dimensional stimulation of excitable tissue can depend on the three dimensional electric field distribution at conjunction of fields and the phase relationships of the electric fields to produce biowave effects. The size of the zone of effect can be defined by parameters, for example the shape of the projected microwave energy distributions and the microwave phase relationships at the crossing point of the distributions.

This microwave field behavior is advantageous in that it can be important in medical therapeutics that neuromodulation effects do not cause undesirable stimulation of excitable neurons at the skin or along microwave paths through tissue; rather only at their conjunction. In this manner, the method and system of the invention can produce activity which can be located within tissue through wave conjunction in a way basically unknown to any other modality for treating excitable tissue.

The bioactive wave at its most basic level of effect, in addition to phase and geometry, is defined by two different aspects of the microwave energy: its carrier frequency and its pulse characteristics. A bioactive single pulse is defined by its envelope amplitude, duration, and how it is modulated. This modulation can be complex with single or multiple frequencies in the range of about 1 kHz to MHz.

As the frequency rises, and in concert with increases in microwave carrier frequency, there is a progressive stimulation of the cell dielectric structures of a smaller size with ultimately the activity of macromolecules being affected at MHz modulation frequencies. Thus microwave pulse modulation is a factor in determining which cellular structures are being activated through this modulation.

Bioactivity involving cell bioelectrical effects such as action events can be achieved when a modulated microwave pulse has a duration so that it produces an effect that exceeds a neural action threshold. Longer than this minimum duration then produces a continued effect that is limited by physiologic accommodation.

For example, a bioactive wave event may result from pulsing the microwave system to produce a one to two millisecond duration that is amplitude modulated in the range of 10 kHz to 1 MHz. The exact frequency can be determined by the acoustical characteristics of the targeted neural membrane structures. These in turn can be defined by the type of cell and the structure of macromolecules forming cell membrane.

By tuning the pulse modulation frequency to the vibrational peak of membrane response, the bioactive effect can then depend on both the microwave strength as well as duration of the microwave pulse. This response dependence on two variables can give rise to bioactive wave strength-duration plots, which show the minimum microwave strength (power density) needed to produce a bioactive threshold event over a range of pulse durations.

The bioactive wave pulse durations and timings may be similar or different compared to electrical stimulations known to the art since the waves offer more variables for control over the stimulation process than does electrical application.

Under the method and system of the invention, bioactive pulses can be selected by electrophysiologists or medical practitioners for amplitude, duration, and repetition rates specific to physiologic effects at the higher levels of the physiology.

For purposes of neuromodulation, physiologic accommodation can be variable. In general the method to reduce this is to keep microwave power level to a minimum that is effective, and by empirical trials in selecting microwave pulse characteristics such as pulse intensity, pulse duration, and pulse repetition rate that allows desirably rapid neural recovery. Accommodation also can be reduced by the method of rapidly moving microwave emission sources. This can be accomplished by moving an actual or virtual microwave source location such that there is a sweeping or redistribution of microwave interaction zones within tissue.

The method and system of the invention has significant advantages over the present medical therapeutic art of affecting living processes by application of electrical current, such present treatments including implanted stimulating electrodes, magnetic pulses, ultrasound energy, and driving transcutaneous currents using skin electrodes. These existing methods have significant drawbacks in terms of their effectiveness, trauma, or related discomforts of pain during stimulation. The advantages of the method and system of the invention can include wirelessness, ability to work through clothing on the skin, no need for electrode or ultrasound coupling agents, and noninvasiveness compared to the use of pulse generators known to the art.

Treatment effects of the method and system of the invention can require a combination of factors, including the microwave carrier frequency needed for bioactivity, pulsed over short durations, applied by way of multiple sources from different directions, having specific modulation frequency and phase relationships and any combination of the foregoing. Treatment effects of the method and system of the invention can be referred to herein as a bioactive wave.

Unexpected neuromodulation and other treatment effects can be produced by a combinational effect of multiple microwave sources of less than 30 GHz providing electric fields that overlap at neural or other excitable tissues in a human body. Neurostimulation and other treatments hereunder by the application of overlapping microwave sources has a number of advantages over current electrical neurostimulation in that there is no required direct contact of electrodes to the skin. Rather various forms of non-contacting antennae or other microwave wave emitting devices can be employed to project energy into the human body through clothing. Additionally, neurostimulation or other treatments of excitable tissue by overlapping microwave fields creates excitatory effects where they overlap. The overlapping fields could be arranged so that they occurs deeper in the body tissue than accessible to electrical neuromodulation by TENS units or applying electrical stimulation to the skin.

The method and system of the invention can be utilized for noninvasively causing a selective alteration in physiologic activity of living things, for example changes in neural bioelectrical excitability, increased metabolic activity, and alterations in the functionality of living tissues. Such method and system can optionally induce physiologic changes by an ensemble of pulsed microwave fields constituting bioactive waves in the effect of the fields on living things. Such method and system can optionally employ bioactive waves as a means to alter the action potential thresholds of nerve, spinal ganglia, and complex neural networks in order to create alteration or a greater facility of cellular neural intercommunication. Such method and system can optionally employ bioactive waves as a means of noninvasively treating pain. Such method and system can optionally employ bioactive waves as a therapeutic device, for example to treat medical disorders of the body responsive to bioelectrical and cellular metabolic stimulation. When utilizing noncontact wave emitting devices, the method and system of the invention does not employ body surface or implanted bioelectrical stimulation electrodes and can thus eliminate the invasiveness of such electrodes and electrode metal contact to tissue. The method and system of the invention can optionally be utilized for cardiac pacing in a manner that does not require contacting electrodes to the heart. The method and system of the invention can optionally be utilized for noninvasive cardiac myocardial stimulation, rhythm support, and therapeutic activation of cellular metabolism useful in recovery from myocardial infarction. The method and system of the invention unexpected provides that microwave energy can be stimulate or treat excitable tissue under certain conditions by the use of overlapping vector beams from microwave sources or wave emitting devices.

### Sample Applications

### Bioactive Wave Device to Produce Physiologic Change

The method and system of the invention can optionally provide a device that produces changes in tissue function through alteration of its bioelectrical or metabolic activity. The device can include at least one microwave energy source that provides two, three or more microwave energy beams of a single frequency having controllable amplitude and phase and whose electric field emissions are directed by antennae or other wave emitters to cross at targeted tissue. The microwave beams may optionally be derived from a single microwave power amplifier and split into multiple antenna with phase control networks on each or employ separate microwave signal chains allowing flexibility in signal synthesis for each antenna.

A triad or other plurality of antenna can launch electric fields to a conjunction within targeted tissues. The electric field vectors emitted from the antenna may optionally all be in a single plane directed at tissues. Other embodiments may optionally employ mutual 90 degree X-Y-Z vector relationships so to alter the volumetric shape of the bioactive region at the target.

The method and system of the invention can optionally employ two, three or more body surface contacting antennae. Such antennae can optionally be of flat form factors, and may optionally use contacting impedance-matched electrodes that employ soft gel dielectric matching layers. A design criterion is optionally provided to create ordered crisscrossing of planar electric field geometries so as to produce an ordered field pattern at the targeted tissue.

The method and system of the invention can optionally use multiple triads or other pluralities of antenna where each antennae group has a different and tilted plane of spatial orientation that provides for a multi-vectored combinational electric field effect conjunct at the targeted tissue. Improved bioactive effectiveness particularly useful for neuromodulation applications that help compensate for unknown cellular membrane orientations is provided.

The method and system of the invention can optionally employ a single microwave source to create the required ensemble of two, three or more crossing fields by reflections, refraction, or scattering such that there appears at a biological target to be multiple wave sources from the crossing directions.

The method and system of the invention can optionally use simple dipole antennae to produce bioactivity, for example when equi-spaced, so as to project their fields inward to cross within a target. Common microwave frequency waveguides and horns may optionally be utilized with appropriate couplers to project through space and through the body surface. These devices may optionally be coupled directly to the body surface through impedance matching layers such as comfortable dielectric liquid or gel bladders interposed on the skin. Adjustment of each antenna tilt and orientation can be optionally provided so that the emitted radiation fields can be easily directed to cross at a specific target depth.

The method and system of the invention can optionally employ forward swept V-shaped loaded dipole, whose rod tips touch a dielectric on the skin, as wave emitters. Common rules of near-field coupling and sub-wavelength antenna theory can optionally be employed with the goal to produce a crossing of wave fronts in tissues that are planar or slightly curved.

Similarly antenna designs can optionally employ adjustable electronic phase delays for purposes of phased array microwave communications. An antenna applicator can be designed in this manner to produce three sources of 120 degree phasing relationship, or other symmetrical designs.

The average microwave power level, which is a safety parameter, can be controlled by the duty cycle ratio. Pulsing of the microwave carrier with suitable on-off duty cycles, for example 1:100 (1 millisecond on with 100 milliseconds off at 10 Hz), can allow higher peak energy for greater bioactive effect while keeping the average energy low. Tradeoffs in the maximal pulse power, because of biologically defined stimulation pulse durations and repetition rates needed for functionality, may limit the possible duty cycle.

In exposed biological preparations where there is no significant path of microwave absorption, bioactive wave effects can optionally be produced at less than 10 W/cm². Deep targets in the human body may require peak power pulses in the 100's W/cm² range in short pulses with lower average power within safety limits.

The base carrier wave frequency may optionally be in the 0.9-5 GHz range, and for noninvasive medical therapeutics may optionally be in the 1.5-2.4 GHz range.

Care should be taken in the selective activation of the cells of the brain because they are in complex interweaving organizations, whereby only the membranes that are in specific orientations to the microwave vector fields are activated. The membrane orientations of dendrites, synapses, glial cells can be unpredictable and to some analysis may be essentially random.

A method and system of the invention can optionally be provided that accommodates the complexity of biological membrane can optionally modulate microwave pulses with two or more frequencies at one time or in rapid sequencing over multiple pulses such that neural structures experience a synergistic effect. Such modulation can optionally be combined with the use of two or more microwave carrier frequencies emitted by two or more triads or other plurality of microwave antenna. A quasi-random modulation may optionally also be imposed in an effort to capture, in a statistical way, a population of cells not having a strong tuning preference. Although less efficient since some energy may be expended in noneffective ways, such modulation technique attempts to capture a statistical average value of excitation.

Two triads of antennae can optionally be applied to brain tissue. One triad optionally operates at 1.5 GHz with a 5 kHz sub-modulation, while the other triad directed at the same target optionally operates at 2.2 GHz with a 20 kHz sub-modulation. The two different triads of microwave source emissions activate different cells within the same target location and so desirably produce a combinational effect that accommodates the complexity of tissue structure and function. Two or more antennae tilted in different orientations can optionally then be sequenced in time with the first triad, which improves the likelihood of engaging other cells not well oriented to the first triad.

### Neuromodulation

Combinations of microwave carrier and modulation frequencies under the method and system of the invention can stimulate physiologic change in different neural structures in the body. For example, for stimulation of peripheral nerve activity each pulse can optionally be AM modulated in the range of about 1 kHz-100 kHz. The preferred modulation frequency can be dependent on whether application is locally to nerve endings, along the nerve axon, at the spinal entry point to the cord, or to the dorsal ganglia. The pulse repetition rate is governed by known neuromodulation protocols and optionally range from a few Hz to typically a few hundred Hz range.

A partial list of such biowave applications include:
peripheral nerves that include for example spinal nerve, vagus nerve, trigeminal nerve, occipital nerve, sacral nerve, peripheral nerve;
sensory organs for pressure, temperature, proprioception, sensitivity to pressure, the eye and ear;
spinal ganglia;
brain cortex, white matter, gray matter, tissues of the frontal, parietal, occipital, and temporal lobes;
brain organs that include for example the hippocampus, thalamic nuclei, endocrine glands;
smooth, skeletal, and cardiac muscle;
cells of the layers of the skin, hair follicles, teeth, gums; and
cellular metabolism and homeostasis.

### Medical Therapeutic Applications for Bioactive Waves

The method and system of the invention can optionally provide a medical therapeutic devices. Such devices can apply bioactive waves to the human body by one or more triads, or other plurality, of microwave emitting devices positioned near or on the body surface whereby their emitted fields are directed inward through the skin to come to a conjunction at a target neural structure or body organ. The bioactivity of these fields can produce physiologic change so as to stimulate neural structures or body organs to affect their function in ways defined by a physician in order to improve health conditions.

Bioactive wave technology offers advantages in that it eliminates implanted pulse generators and their silicone lead systems used to treat bioelectrical based disorders known to the medical arts. Treatment of such disorders by neurostimulation techniques known to the art at this time include muscular rehabilitation, epilepsy, Parkinson's disease, obesity, migraine, mood control, arthritis, neuralgias, nerve conduction issues, and disorders of the gut. In a similar way, bioactive wave neurostimulation can be applied noninvasively to the therapeutic target sites of the foregoing disorders that are within range of microwave energy penetration.

The medical application of bioactive waves can be both therapeutic as well as diagnostic. Diagnostic applications can optionally be based on the nature of a physiologic response, or lack of, for an organ to an applied bioactive stimulus. For example, a physician would place the microwave source field crossing directed at the intended target organ or at nerves leading to the organ. There would be followed by a search for the best combination of microwave setup parameters that produce the intended effect. This could be based on the physician's prior experience or that of others with the device settings, or if a first attempt, through a process of stepwise progression through a range of parameters until the desired response is obtained.

A method of determining the settings of bioactive parameters when their specific ranges are unknown would be to employ stepwise strategies to converge on optimal settings. For example, an optional strategy would be to first set the pulse width to a nominal 5 milliseconds, a pulse repetition rate of 1 Hertz (200:1 duty cycle), fix the microwave carrier frequency at 1.5 GHz as a nominal value, set the power level to a nominal peak pulse value of 200 W/cm² (1 watt/cm² average power) and then move in steps of 0.1 GHz (ten steps) to 2.5 GHz. At each step the pulse modulation frequency would then be scanned through a range for example of 1 kHz to 100 kHz taking several seconds for each step. A physician would observe responses and listen to patient reports of localized feelings of pressure, a pulsing sensation, a prickly sensation, or transient sensation of warming.

Other effects depend on the placement of microwave applicator and include for example, relief of pain when applied to the nerve near the pain site or at or near the nerve's entry to the spinal cord. Finer steps, expanding the frequency range, or adjustment of power may be selected if results are not satisfactory.

Similarly there are a range of operating parameters that in combination with specific placement of the bioactive wave device on the body can produce alterations in heart performance when placed on the chest directed at the heart, changes in cognitive functioning when applied to the brain, sensory effects when applied to sense organs, motor responses when applied to anywhere in the neural path activating muscle. All of these effects would be directed by a physician to achieve therapeutic goals.

### Vagal Neurostimulation

The method and system of the invention can optionally provide a noninvasive device for vagal nerve stimulation, for example as a means of treating multiple medical disorders including epilepsy, arthritis, sleep disorders, and an increasing number of disorders of the lower gut. Vagal neurostimulation under method and system of the invention can optionally be achieved by applying the bioactive wave to the side of the human neck region over the vagus and selecting bioactive wave stimulation parameters according to therapeutic goals.

For example, the vagus is composed of a range of fiber diameters and cell membrane morphologies. These fibers are known to fall into classes such as large myelinated A fibers, smaller but still myelinated B fibers, and small unmyelinated C fibers. Unfortunately there is no known effective way known to the art to stimulate specific fibers for the therapeutic control of indigestion or epilepsy without undesirably also affecting its branches that go to the heart or vocal cords.

Under the method and system of the invention, a greater selectivity of vagal neurostimulation effect can be achieved by tuning both the microwave carrier frequency and the pulse modulation frequency to produce effects tuned to specific A,B,C classes of its neurons. This tuning effect derives from the different best vibration frequencies that are a function of the different axon morphologies. Such frequencies would vary depending on their function, such as sympathetic, parasympathetic, sensory, or control. These differences are governed, for example, by the mechanical relaxation time constants of tissue depending on its elasticity and variation in the physical structure or organization.

Thus it is possible by the tuning of bioactive wave modulations of the lower frequency range of 1 kHz-50 kHz to be more selective to A fibers in a nerve bundle for example, without necessarily stimulating C fibers at higher frequencies. In this manner, the method and system of the invention provide a powerful tool allowing a much more specific and finer control of neuromodulation by bioactive wave techniques. Resulting trauma to tissue is less since the method and system of the invention offer a great degree of selectivity and range of therapeutic effects. The noninvasiveness of the method and system of the invention may obviate the need for surgical implantation of a cuff electrode to the nerve.

Bioactive waves with previously selected frequency modulations so as to stimulate specific nerve actions can optionally be then further customized in effect by pulse duration adjustments, for example, from 0.1 millisecond to as much as 50 milliseconds, and pulse repetition rates, for example in the range of about 2 pulses per second up to greater than 1500 per second at short pulse widths, depending on preferences.

### Pain Relief

The method and system of the invention can optionally provide a bioactive wave device that relieves pain, for example arising in conditions that are currently being treated with TENS units. Bioactive waves can be applied to the region of the skin, including for example where TENS electrodes are currently applied, such that they stimulate nerve pathways known or otherwise effective in providing the competitive masking sensation that block pain. Pain relief accomplished by bioactive wave techniques can be more effective in this situation since skin electric shocking that occurs with high settings of TENS electrical currents is avoided.

For example, for the relief of pain by driving near surface neural responses, bioactive microwave carrier frequencies can optionally be adjusted to the less penetrating higher end of the range of 2 GHz to engage neural circuits, for example those currently stimulated by TENS units. The pulse modulation frequencies can be adjusted over any suitable range, for example in the range of 1 kHz to 1 MHz, according to patient reports of sensation. The pulse durations and repetition rates can optionally be similar to those known to those skilled in the art of neuromodulation, for example in the range of 0.1-100 milliseconds, pulse repetition rates can optionally be in the range of 1-1500 per second, and amplitudes selected at a level of pain relief that does not drive other side effects such as muscle stimulation. The desired pulse protocols are selected by the patient, under supervision by a physician, so as to maximize relief.

A starting point in the microwave power application can optionally be, for example, an average value 0.1 W/cm² and then increased to higher levels for effect as determined by the physician and patient. At some level the tingling sensations felt by the patient become stronger to a point reached where the patient is satisfied with the level of pain relief. The microwave total energy rate delivered can optionally be limited by safety, but can optionally be around one to two watt/cm² average power.

### Bioactive Wave Spinal Cord Stimulation (SCS)

The method and system of the invention can optionally be utilized to provide pain relief by applying a biowave to a patient's back over the spinal region. An initial placement strategy of the antenna or other wave emitting device application can optionally be the same spinal location where stimulating electrodes are currently placed for implantable spinal cord stimulation (SCS) devices. This location can optionally be above the entry point of the nerves to the spine that originate at the point of pain.

Pain relief can optionally be provided by directing the bioactive wave to the lateral spinal ganglia such as the dorsal root ganglia. Multiple bioactive wave devices may optionally be used on both sides of the spinal cord region for the relief of pain. Since these ganglia are less accessible to current electrical techniques of pain relief because of physical limitations of accessing then through the ribcage, the biowave application by the method and system of the invention enables noninvasive therapeutic approaches not otherwise possible.

In some cases upon choice of the physician, pain can optionally be relieved by applying the bioactive antenna to acupuncture points corresponding to specific nerve plexus at the choice of the physician.

The methods and systems of the invention applicable to pain relief can optionally, and advantageously, be implemented by portable versions useful on-demand by patients for manual application to accessible parts of the body where pain originates.

### Bioactive Wave Cardiac Therapeutics

The method and system of the invention can optionally be utilized as a bioactive wave heart stimulation device for purposes of pacing and correcting rhythm disturbances. The biowave applicator can optionally be positioned on the chest wall surface. The microwave fields can be directed to cross at a chosen location in the heart so as to bioelectrically stimulate its susceptible tissues.

Particularly susceptible are those tissues which are the origin of electrical activation of the heart, for example the S-A or A-V nodal tissues, and to a lesser extent the cardiac Purkinje system known to the art of medicine. Bioactive waves can provide a degree of localized activation of the heart.

Pulses of microwave energy, for example from three equi-spaced sources in a triangle, can optionally be projected inward such that their fields cross and converge at the desired excitable site on the heart as chosen by medical personnel. The actuation of the heart can optionally be achieved by pulsing the system periodically at the desired heart rhythm, for example 72 beats per minute.

For cardiac application in adults, the microwave frequency can optionally be chosen at the lower end of the 0.9-2.2 GHz region to achieve greater chest wall penetration, while in children its frequency can remain at the higher and more effective end due to a shorter radiation transit path length.

The pulse modulation characteristics can be in any suitable range, for example as disclosed herein. The pulse durations can optionally be in the range of 0.1 millisecond to approximately 10 milliseconds duration, depending for example on microwave power applied and on the health of the heart. Longer pulses may be needed with weak heart function. The microwave power can optionally be adjusted to achieve desired strength of cardiac effects, depending for example on the patient's body mass affecting microwave path losses. The pulse power can optionally be relatively high, for example at 100's watts/cm² over short pulse bursts, and limited in average power by safety standards.

Each pulse can optionally be modulated by a frequency in the range of 100 Hz to 100 kHz, for example consistent with its pulse duration. Modulation of the applied pulse can optionally be used to contribute to specificity of cardiac responses. Myocardial cells are not all the same and their morphology is dependent on their function. Accordingly, the pulse modulation frequency can optionally be chosen to resonate with specific types of cells, for example as determined through a stepwise search as described earlier. Therapeutic responses are determined by a physician in achieving the desired effectiveness, including for example at minimum power consumption and tissue heating.

An advantage of the noninvasive aspect of the bioactive wave stimulation of the method and system of the invention is that it provides a new method for direct activation of the left heart, which is less difficult than current pacemakers that require specialized surgical techniques to achieve. Left heart synchronization to the right heart rhythm by bioactive wave can optionally be achieved, for example utilizing the method of closed loop feedback based on an ECG or other measures of cardiac function to provide best heart function.

In a similar manner, arrhythmias such as but not limited to atrial tachycardias may be treated by externally applied bioactive waves of the method and system of the invention synced to the ECG timing and directed to myocardial activation at the heart nodal sites.

An advantage of the method and system of the invention is that noninvasive transthoracic pacing can be achieved by adjusting the emitted radiation patterns to substantially cross within the heart avoiding the stimulation of the skin pain receptors and other tissues of the chest and pleura along the path. Another advantage of application of the method and system of the invention to the heart is that the bioactive wave volume of tissue stimulation can be extended to engage a larger region of tissue than can be achieved by a single point cardiac electrode. This allows a larger zone of heart stimulation effect and gives the physician a new strategy for management of heart rhythm disorders which do not rely on an electrode point of activation with a propagating wave. Rather there can be a more global activation of the whole heart which a physician may find valuable in defibrillation, resuscitation, treatment of cardiac arrest, and in continuing life support while not requiring contacting electrodes to drive currents through tissues.

### Bioactive Wave Treatment of Myocardial Infarct

The method and system of the invention can optionally be utilized as a device used to treat the sequelae of heart attack, known as myocardial infarction. This condition is due to blockage of a heart blood vessel resulting in the deprivation of blood and eventual death of heart muscle in a local region. The irretrievable death of muscle however takes as much as hours or days usually surrounded by heart muscle that is marginally alive but not contractile.

Bioactive wave treatment under the method and system of the invention can reduce the region of muscle death by way of strongly stimulating heart cells to respond; more so than would naturally occur. A strong bioactive wave stimulus to the heart cells to contract where they otherwise may not due to their debilitated condition can improve cardiac output, increases blood flow by collateral flow into the surrounding blocked-flow region, and so allows ischemic tissue to recover function and reduce the ultimate size of the infarcted region and improve patient outcomes.

Under the method and system of the invention, a bioactive wave device can optionally be applied to the heart region and either set to pace or alternately synchronized with intrinsic heart rhythm by ECG recording. Pulse parameters of the bioactive wave can then be optimized, for example as described herein for in other neurostimulation applications, for either direct stimulation of the Purkinje pathways or for stimulated contraction myocardial cells surrounding the ischemic region. Such treatment can be quasi continuous or applied periodically as the heart function recovers over time.

### Implantation of Bioactive Wave Generators

The method and system of the invention can optionally provide an implantable device that produces the needed conditions for bioactive waves from implanted antennae. Such a system or device can optionally be in the form of small microwave generator system in a battery powered package similar in size and construction to current pacemaker devices. The device can optionally have an antenna or other wave emitter that is integral to the package and projects energy outward. The method and system can optionally include one or more silicone coated antenna leads, or other wave emitters, that provide a triad or other plurality of microwave emission sites that are strategically placed by surgery so as to produce the desired crossing of fields at a target body neural system or organ.

Bioactive wave stimulating catheters or devices, such as under the method and system of the invention, are superior to conventional electrode implants in that there is no electrode-tissue interface that conducts a current flow. This eliminates problems, for example, of in-vivo electrode degradation, local tissue pH changes, and hermeticity of electrode leads.

The microwave emitters within the encapsulation of the catheter or device of the invention can optionally be arranged by a strategy to produce bioactive effect at some distance from their collective emission sites where the individual wave phases overlap. For example, three or another plurality of microwave emitter points separated by some distance within the length of the catheter can optionally be provided.

An advantage of such a method and system is that there is no need to continually wear an external device and elimination of the corrosion and deleterious effects of localized high density electrical currents applied by metal electrodes conducting currents to tissues.

Such a catheter method can optionally be utilized for brain applications where an ability to electronically move the site of activation by microwave amplitude and phasing control, can provide new therapeutic modalities. Such a method and device can allow for selective targeting of specific neural structures that may be too close together to separate cleanly by way of the larger extent of the bioactive zone produced by microwave frequencies needed to penetrate from the skull surface.

A number of applications and implementations of the bioactive wave technology of the invention have been described. Such technology can provide patterns of electrostrictive force to create effects at susceptible cellular scale structures. Such technology can also serve as a method of scientific investigation of microscale phenomena in dielectrics and in the development of microwave-based tools to the understanding of cellular physiology.

As a foundational technology using a microwave based acoustic method of interacting with the cellular bioelectrical and biochemical nature of living tissues, rather than current electrical current methods, the technology can be implemented in different ways both in its electronic device design as well as variation in the method of application. Various modifications of the technology and its applications can be provided and still be within the scope of the invention.

### Discussion of Drawings

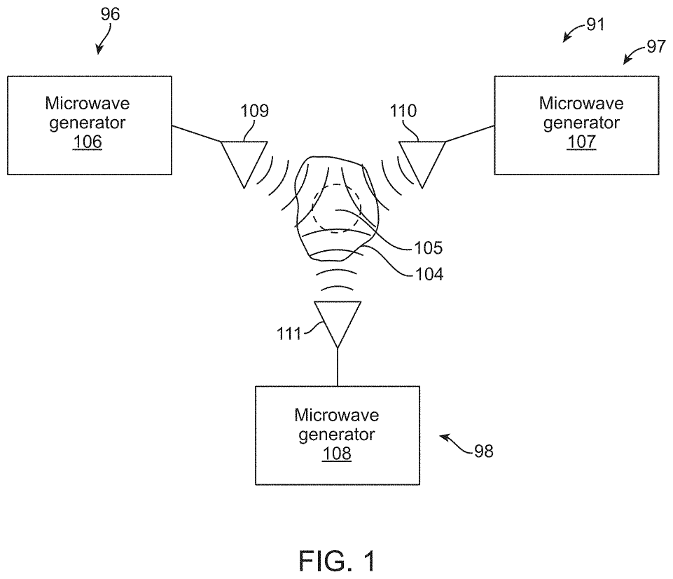

An embodiment of a method and system of the invention is illustrated in FIG. 1. System 91 therein includes at least one microwave source for providing a plurality, for example three, bioactive waves. The at least one microwave source include a system of three microwave sources, specifically first microwave source 96, second microwave source 97 and third microwave source 98. Each microwave source can be of any suitable type. Sample first microwave source 96 includes a respective first microwave generator 106 and a suitable wave emitter in the form of first antenna 109. Sample second microwave source 97 includes a respective second microwave generator 107 and a suitable wave emitter in the form of second antenna 110. Sample third microwave source 98 includes a respective third microwave generator 108 and a suitable wave emitter in the form of third antenna 111. The antennae 109-111 emit respective microwave energy, in the form of a microwave beam, that penetrate into a biological object 104 and cross at target position or bioactive zone 105 with the biological object 104. Bioactivity occurs most strongly at the cross point 105 of the respective vector electric fields, that is where the three microwave energy beams overlap or intersect, where treatment is desired. At other positions within the biological object there is not the needed combination of conditions to produce bioactivity. For example, bioactivity is much reduced along the electromagnetic field path of each microwave and does reach a threshold until approximately the midpoint of the beam crossings. This is of advantage in comparison to electrical stimulation of bioactivity whereby the strongest electrical effects are generally close to the stimulating electrode.

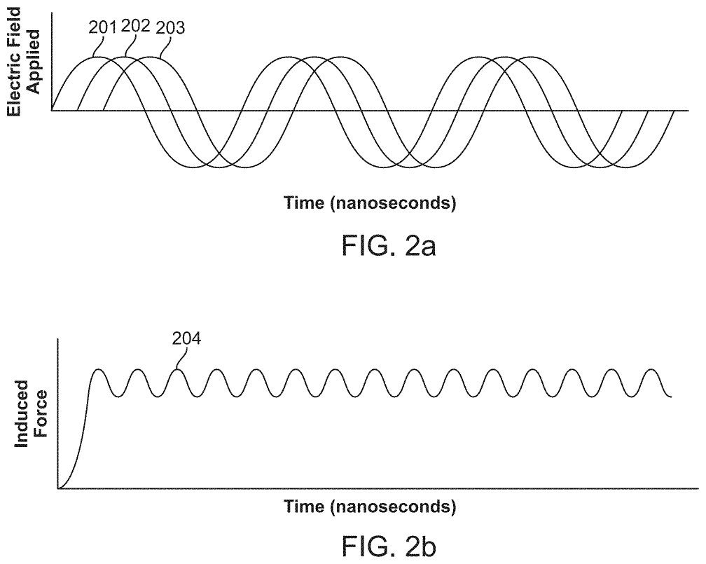

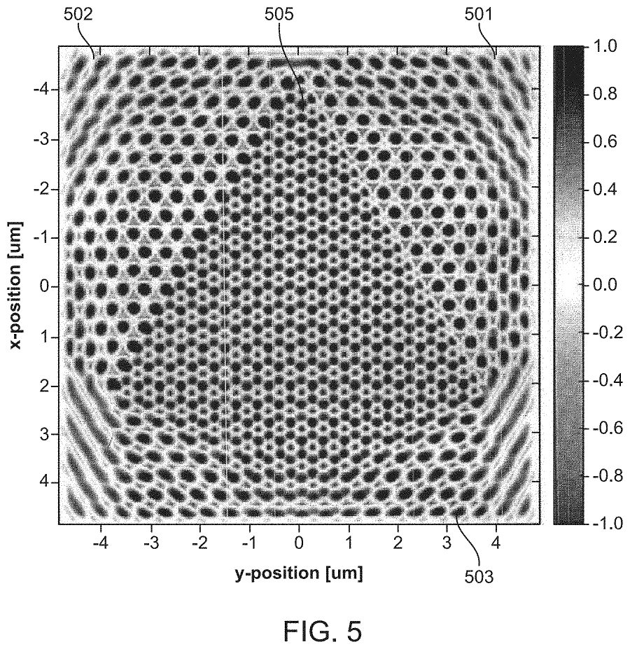

In FIG. 2a, there is shown the time course of three oscillating electromagnetic fields 201, 202 and 203, for example respectively produced by the three antennae 109-111, in the microwave frequency region. The three fields can optionally be of the same frequency, but out of phase from each other as shown in FIG. 21. When three 120 degree phased waves, for example the three microwaves produced by antennae 109-111, are coincident in a dielectric there is produced a resultant force waveform 204, for example illustrated in FIG. 2b. As discussed above, the resultant force is proportional to the sum of the squared value of the three or other plurality of electric fields. In part there are hypersound frequency harmonics generated resulting from the nonlinear electrostrictive squaring effect as well as a sustained average force that produces an offset from the baseline. The overlapping effect creates constructive and destructive interference between crisscrossing hypersound waves, for example from originating from 120 degree angles. This interference of peaks and nulls of acoustic force over tens of nanometer-order distances produces a type of moiré grid of hypersound force cells. In tissue, this pattern of forces can penetrate and appear within its volume and can cause shearing on cellular membranes. The foregoing effect is shown in FIG. 5 whereby a computer model shows the result of three plane waves sources arranged in a triangle around the edges of the volume. These sources are modelled as having a hypersonic wavelength of about one micrometer. They propagate from the periphery and then overlap in the space between them showing the moiré pattern of wave interferences.

The moire pattern of hypersound wave intersections can create a grid of high force gradients at the tens of nanometer scale. These forces can distort the shapes of cellular membranes having features that are on this scale of size. There can be a grid of twisting and shearing effects created by the crisscrossing of hypersound waves that can create distortions of the cell ionic channels in membranes. This distortion can be considered to alter cellular ionic kinetics, such as sodium, potassium, or calcium channel transport, leading to shifts in cellular transmembrane potential and ultimately action events of excitable neural or other tissues.

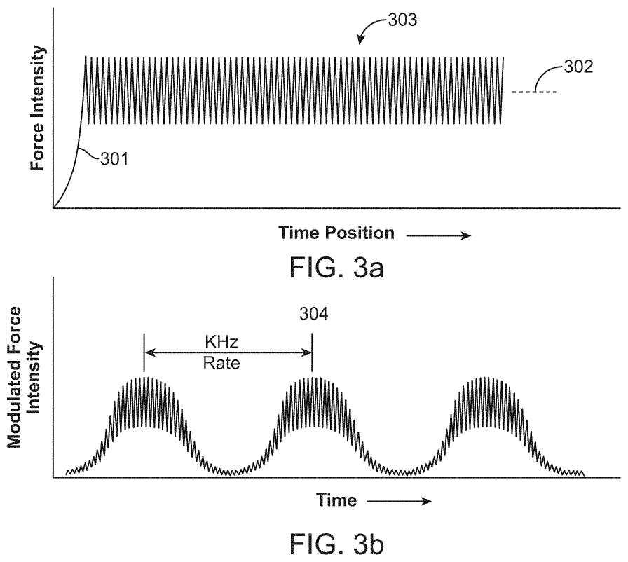

FIG. 3a is a representation of the effects of microwave-induced electrostrictive force action on a different time scale showing again the production of a steady average force 302 with a high frequency secondary hypersound wave 303 riding on its steady pedestal wave 301. The waves of FIG. 3 are the same waves as in FIG. 2 but on a different millisecond time scale that is much longer and shows the overall effect of modulation on the microwave carrier. Wave 303 in FIG. 31 represents hypersound forces. The force intensity in FIG. 3a can have the same meaning as the induced force in FIG. 2b. FIG. 3b shows on this different scale, for example in milliseconds, the result of the amplitude modulation of the microwave electric fields at typically kHz to MHz rates 304. There can be an additional and lower frequency of vibration in the kHz to MHz region generated as an effect of the microwave modulation. Thus the electrostrictive effect of producing a steady force pedestal on which hypersound forces appear can itself be chopped by the modulation of the microwave producing lower frequency vibrations on cell membranes. FIG. 3b shows pulses of hypersound vibration that are riding on a lower frequency modulation. The result is the relatively lower frequency pulsing of forces in the kHz-MHz range that are made up of hypersound vibrations summed with them. The overlapping microwaves of the method and system of the invention can provide a moiré pattern of induced hypersound forces on a direct current offset that can then be pulsed producing low frequency Fourier components in kHz-MHz range. Bursts of these can be crafted by the physician for specific physiologic effects. The lower frequency modulation on GHz microwaves is an important feature of the invention, and bursting of the modulations can produce specific physiologic effects on the excitable tissue, for example nerves.

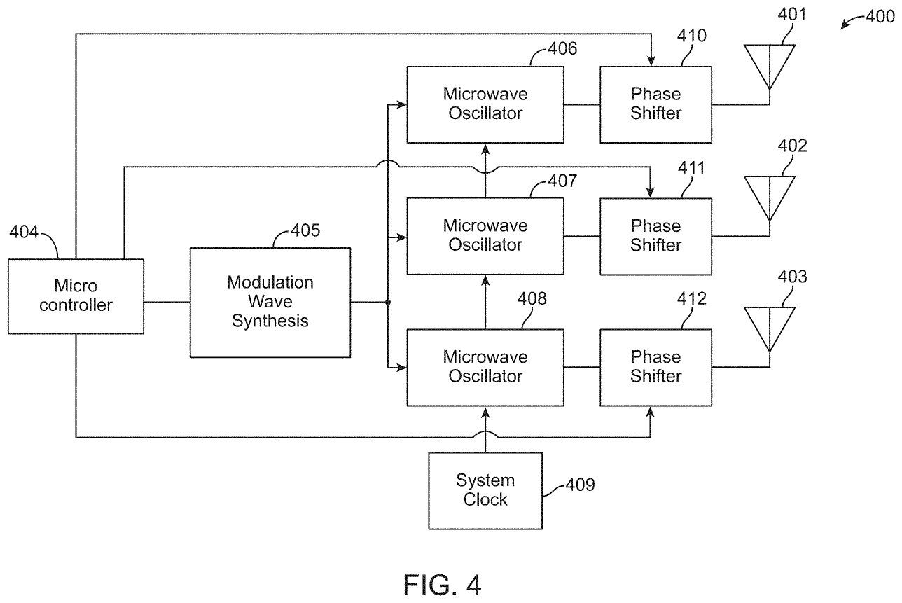

FIG. 4 illustrates a sample microwave generator system 400 of the invention that employs a design using three separate microwave sources with digital control over the phases. It consists of three microwave emitters, in the form of first antenna 401, second antenna 402 and third antenna 403, a microcontroller 404, a modulation wave synthesizer 405, first microwave generator or oscillator 406, second microwave generator or oscillator 407 and third microwave generator or oscillator 408, each phased to a common clock 409 and respectively driving a phase control network that includes first phase shifter 410, second phase shifter 411 and third phase shifter 412. System 400 is able to generate AM modulated microwave frequency pulses of variable width, duration, repetition rate for use in the method of the invention.

FIG. 5 illustrates the output of a computer model showing, by gray scale, the amplitude of forces as a function of position generated by the interaction of three 1 GHz hypersonic wave fields 501, 502 and 503, for example produced by system 400. The fields intersect in the central zone from 120 angles to produce a triangular moiré grid 505 of wave interactions defining grid of nanoscale force cells of compression and rarefaction. The forces experienced by cellular dielectrics are greatest where there is a shearing action between adjacent cells undergoing opposite directions of compression and rarefaction. Such moire pattern of crisscrossed hypersound forces has the darker zones of compression and the lighter areas rarefaction in FIG. 5. The zones of greatest cell shearing are midway between these two regions.

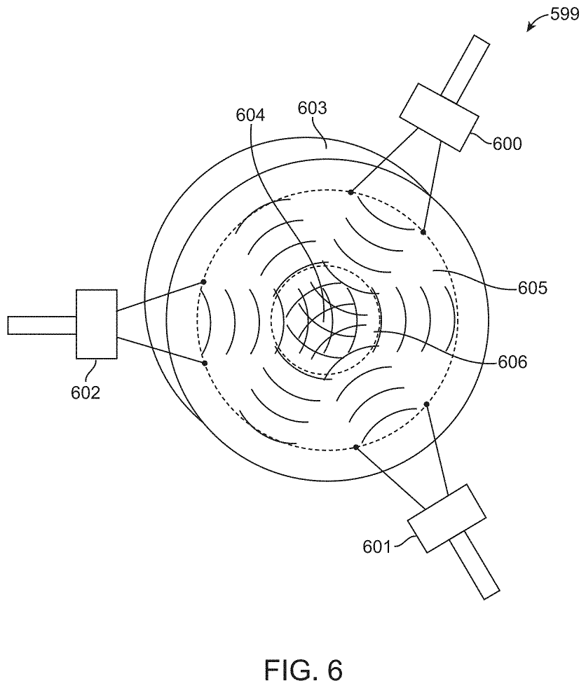

FIG. 6 illustrates a sample microwave antenna applicator 599 suitable for use in the method and system of the invention. The applicator 599 includes a ring 603 that supports sample first, second and third three dipole antennas 600, 601 and 602, or other suitable wave emitters. The applicator 599 can be part of the at least one microwave source of the invention. The antenna elements are tilted downward towards the biological tissue 605 and their field crossings within tissues form the bioactive wave zone 606 within the tissue. Each antenna is shown with a balun transformer so as to insure a symmetric electric field production into the lumen needed to create the needed crossing wave geometries 604.

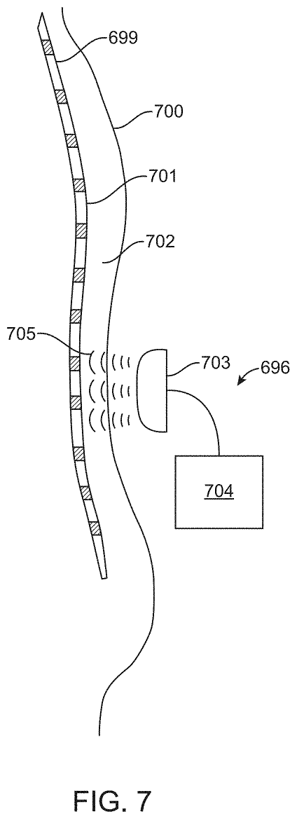

FIG. 7 illustrates a sample application of a sample bioactive wave system 696 of the method and system of the invention to the spinal area of the back of a patient for treatment of pain. Shown is a sagittal view of the human body torso with a spine 699, the back skin surface 700 overlying the spine 699, the spinal vertebrae 701 subcutaneous fat layer 702, and placement of a microwave antenna applicator 703 fed by a bioactive wave generator 704. The applicator 703 and wave generator 704 can be part of the at least one microwave source of the invention.

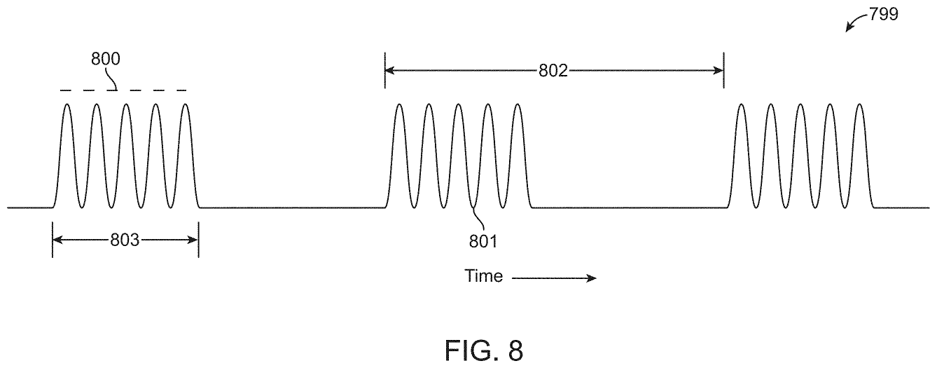

FIG. 8 illustrates a generalized pulsed waveform 799 formed from a suitable method and system of the invention. The waveform has a frequency 801, for example in the kHz to MHz range, used to modulate the high frequency GHz range microwave carrier so to achieve bioactivity. The waveform can optionally be a repeating burst of oscillatory waves characterized by an amplitude 800, frequency 801, pulse period 802 and pulse width 803. These characteristics are chosen for their effect on higher levels of the physiology and organization of biology functionality. The waveform illustrated in FIG. 5 is representative of the envelope of both microwave and hypersound forces from a single antenna or other wave emitter. The five cycles show, for example, are a composite of thousands of cycles of GHz frequency oscillations in a burst from a single wave emitter. The frequency of pulses with a burst, the burst duration, and the intervals between bursts are variables and specific to the specific tissue or organ that is the target of treatment.

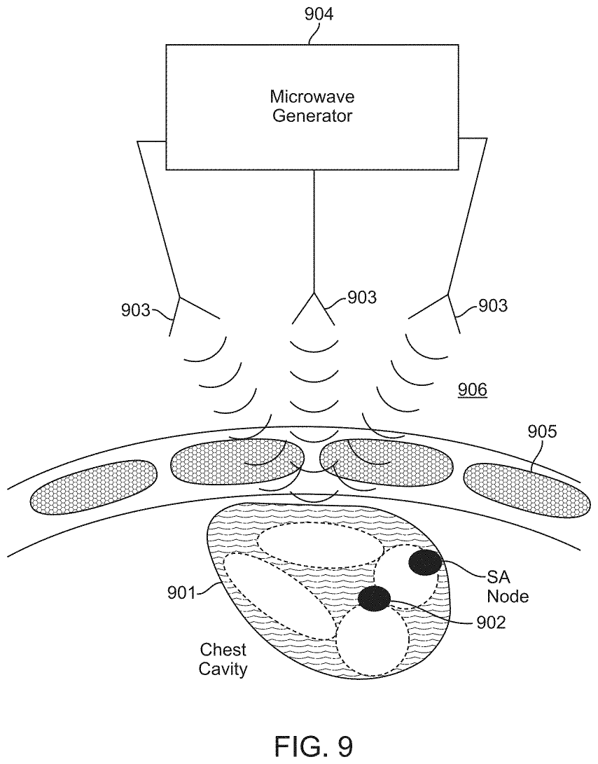

FIG. 9 illustrates a sagittal sectional view of a human body torso showing the heart 901, the ribs edge-on 905, the excitable tissues of the atrioventricular node where fields cross 902, the microwave generator 904 and three microwave antennae or other suitable wave emitters 903. Generator 904 and wave emitters 903 are part of the at least one microwave source of the method and system of the invention. The EM waves 906 launched from the antennae toward the heart propagate through the skin and ribcage to come to a conjunction in the heart. Intervening tissue, that is between the heart and skin of the patient, is not treated by the overlapping portion of waves 906.

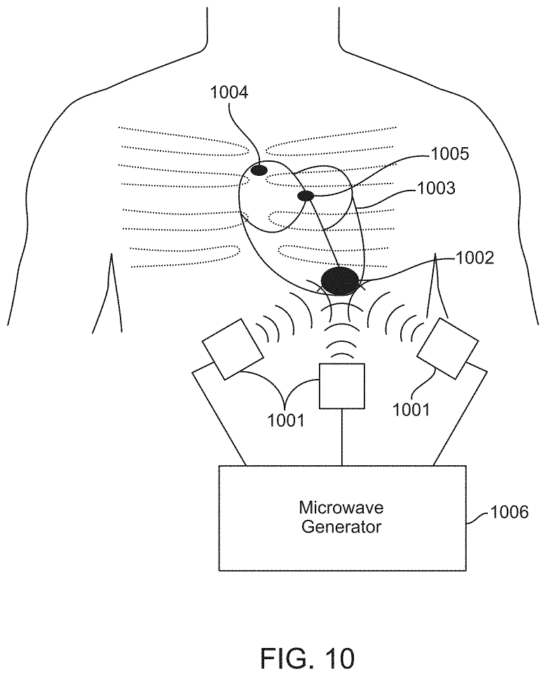

FIG. 10 illustrates three microwave antennae or other suitable wave emitters 1001, driven by a microwave generator 1006 and directed to a region of a myocardial infarction 1002 within the heart muscle 1003 of a patient. The generator and wave emitters are part of the at least one microwave source of the method and system of the invention. The microwaves and respective vector electric fields cross at the node 1005, producing a zone of bioactivity at the node for therapeutic purposes. The field crossings may alternately be redirected to the sinoatrial node 1004 or the atrioventricular node 1005 for heart rhythm control for treatment at those areas of the heart.

In an aspect of the invention, a bioactive wave emitting device or system for altering physiological function of a biological target can optionally be provided that includes at least one or a plurality of electromagnetic microwave sources, a means of application of said plurality of microwave sources to said biological target so as to produce electric fields configured to produce crossing of their electric field vectors at a biological target, and a means of adjusting the operating parameters of each microwave source comprising frequency, power, phase, pulse duration, repetition rate, and pulse amplitude modulation, whereby the crossing of said plurality of electric field vectors emitted by said means of application producing said electric fields configured according to a set of said parameters, directed at said biological target produces said altering of physiologic function.

Each of the at least one or plurality of microwave sources can optionally have a frequency in the range of 300 MHz to 300 GHz, for example in the range of 0.9-6 GHz. The bioactive wave emitting device or system can include means of application comprising the projection of three said electric field vectors from sites at the corners of an equilateral triangle inward towards the central enclosed zone and whose said electric field vectors are oriented to produce said crossing within the space of said biological target. The bioactive wave emitting device or system can include means to apply the electric field vectors to a biological target whereby the means is selected from the group consisting of wave emitting devices, microwave dipolar antennae, and microwave waveguides, and skin contacting electrodes, and antennae employing dielectric matching layers to the body surface, and wholly tissue-implanted antenna-electrode systems. The bioactive wave emitting device or system can include a means of application of said microwave sources comprising a plurality of functionally associated triads of microwave sources related by phase whereby each triad is positioned with other functionally associated triads in a three dimensional configuration with each triad then energized in sequence; whereby there is a greater diversity of said electric field vector directions at the said biological target producing greater effectiveness.

The electric field vectors can optionally be configured as a triad and have relative said electric field phases at the target with respect to each other of 120 degrees. The microwave pulse durations can optionally range of one microsecond to continuous and from 100 microseconds to 100 milliseconds. The pulse amplitude modulation can optionally be in the range of approximately 100 Hz to 100 MHz and from one kHz to two MHz for neuromodulation as consistent with the said pulse duration. The microwave pulse repetition rates can optionally be in the range for neuromodulation effects and can optionally range from one Hz to two kHz, and can optionally be limited by consistency with no overlap of said pulse durations. The microwave power can optionally be set by observation of sufficiency of said alteration of physiologic function, optionally being pulsed within the ranges of 10 mW/cm2 to approximately 10 kW/cm2 or within the range of one W/cm2 to approximately two kW/cm2. The at least one or plurality of electromagnetic microwave sources can optionally drive a triad of antennae, for example tuned to a different carrier frequency, so as to allow physiologic change in two or more cell types or alternately two or more different cellular functions of the same cell type at the same time. The pulse amplitude modulation may optionally comprise two or more frequencies to induce physiologic change in two or more cell types or alternately two or more different cellular functions of the same cell type at the same time, so that a complex response of the living system is achieved that is not otherwise feasible with a single modulating frequency.

The microwave parameters can optionally be configured to achieve desired changes in said physiologic function, and can optionally include the steps of setting microwave source parameters to an initial state per any of the above, stepping one microwave source parameter over a range while sweeping the range of a second source parameter for each step, observing physiological change resulting from the first parameter step during each second parameter sweep, and repeating until desired said physiologic change occurs and identifying these values, whereby identified said microwave parameters according to their physiologic effect are noted and used as reference settings for future applications.

In an aspect of the invention, a bioactive wave emitting device can optionally be configured to effect a physiologic change such as alteration of functionality of target body organs that can optionally include spinal nerve, vagus nerve, trigeminal nerve, occipital nerve, sacral nerve, peripheral nerve, sensory organs such as pressure, temperature, and proprioception, sensitivity to pressure, neural components of the eye and ear, excitable neural ganglia, brain cortex, white matter, gray matter, tissues of the frontal, parietal, occipital, and temporal lobes, brain organs of hippocampus, thalamic nuclei, endocrine glands, smooth, skeletal, and cardiac muscle, cells of the layers of the skin, hair follicles, teeth, gums, vascular blood flow, cellular metabolism, and homeostasis, so as to achieve a desired physiologic change affecting the function of the target organ.

In an aspect of the invention, a method of employing a bioactive wave emitting device, according to any or all of the above, can optionally be provided to achieve desired alterations in the physiologic function by configuring the microwave parameters to produce effects that can include initiation of action events, neural subthreshold bioelectric changes, changes in spontaneous rate of cell bioelectrical firing, alteration of muscle contraction so as to achieve one or more of desired physiologic change.

In an aspect of the invention, pain relief device, that includes any bioactive wave device of the invention, can optionally be applied to the vertebral region of the back and surrounding spinal ganglia regions with the microwave electrical field amplitudes adjusted to a level defined by the user whereby there is produced a reduction of pain and consequent therapeutic effect.

In an aspect of the invention, a method of producing medically therapeutic neuromodulation can optionally be provided that includes identifying the desired therapeutic effect and the body target organ, directing overlapping electric fields to the chosen target organ, setting the microwave parameters in accordance with any of the above, observing for evidence of said therapeutic effect, determining from these steps a protocol of patient use, whereby the physiologic activity altered by the bioactive wave of the invention produces the desired medical therapeutic effect.

In an aspect of the invention, a noninvasive cardiac pacemaker for controlling the rhythm of the heart can optionally be provided that includes a bioactive wave device, according to any or all of the above, applied to heart regions of sinoatrial node, atrioventricular node, or cardiac myocardium and then pulsed at a repetition rate in the range of physiological rhythms so that the bioactive wave pulse repetition rate captures the heart rhythm and becomes the pacesetter.

In an aspect of the invention, a cardiac therapeutic device for improved recovery after myocardial infarction can optionally be provided that includes a bioactive wave device, according to any or all of the above, whose electric field vectors are directed to crossing at and around the infarction and adjusted in said operating parameters to produce physiologic change at the cellular level so that there is an increase in functionality of cardiac cells that can include, but is not limited to, increasing myocardial blood flow through vasodilation and arrest of arrhythmias and aberrant beats, so that the bioactive wave causes improvements in myocardial cell living function.

In an aspect of the invention, a cardiac rehabilitation device for use after infarction can optionally be provided that includes a bioactive wave emitting device, according to any or all of the above, for application to the region surrounding said infarction so as to effect physiologic change that can include, but is not limited to, increased bioelectrical activity, improved tissue metabolism, and increased blood flow leading to reduction of extent of ischemic tissues that might otherwise undergo necrosis.

We claim:

1. A method for treating a patient, comprising providing at least one microwave source capable of producing first microwave energy with a first electric field and second microwave energy with a second electric field, directing the first microwave energy towards excitable tissue within the patient and directing the second microwave energy towards the excitable tissue at an angle from the first microwave energy, wherein the first electric field overlaps with the second electric field at the excitable tissue so as to alter a physiological function of the excitable tissue.

2. The method of claim 1, wherein the directing step includes directing the microwave energy from a first position spaced from the patient and directing the microwave energy from a second position spaced from the patient.

3. The method of claim 1, wherein the at least one microwave source includes a wave emitting device selected from the group consisting of an emitting field element, a microwave field generator, a microwave dipolar antenna, a microwave patch antenna, a microwave waveguide, an antenna employing dielectric matching layers to the body surface of the patient, an exposed coaxial cable, a skin contacting electrode and a wholly tissue-implanted antenna-electrode system for directing each of the first microwave energy and the second microwave energy.

4. The method of claim 1, wherein the directing step includes directing each of the first microwave energy and the second microwave energy at a frequency range selected from the group consisting of 300 MHz to 300 GHz and 0.9 to 6.0 GHz.

5. The method of claim 1 wherein each of the first microwave energy and the second microwave energy is tuned to a different carrier frequency.

6. The method of claim 1, wherein the angle ranges from the group consisting of 10 to 80 degrees and 45 to 80 degrees.

7. The method of claim 1, wherein the at least one microwave source is capable of producing third microwave energy with a third electric field, further comprising directing the third microwave energy towards the excitable tissue at an additional angle from the first microwave energy that is different than the first-named angle, wherein the third electric field overlaps with the first electric field and the second electric field at the excitable tissue.

8. The method of claim 7, wherein each of the first-named angle and the additional angle is selected from the group consisting of 10 to 60 degrees and 30 to 60 degrees.

9. The method of claim 1, wherein the excitable tissue is selected from the group consisting of a nerve, a spinal nerve, a vagus nerve, a trigeminal nerve, an occipital nerve, a sacral nerve, a peripheral nerve, a sensory organ, an organ for sensing pressure, an organ for sensing temperature, an organ for sensing proprioception, an organ having sensitivity to pressure, a neural components of the eye, a neural component of the ear, an excitable neural ganglia, a brain cortex, white matter, gray matter, a tissue of the frontal lobe, a tissue of the parietal lobe, a tissue of the occipital lobe, a tissue of the temporal lobe, a brain organ, a hippocampus, a thalamic nuclei, an endocrine gland, muscle, smooth muscle, skeletal muscle, cardiac muscle, skin cells, hair follicles, teeth, gums, vasculature, a region of the heart, a sinoatrial node, an atrioventricular node, a myocardium and any combination of the foregoing.

10. The method of claim 1, wherein the excitable tissue is a region of the heart, wherein the microwave energy of the first and second microwave sources is pulsed at a repetition rate in the range of physiological rhythms and the alteration of a physiological function is the capture of an irregular heart rhythm similar to a pacemaker.

11. The method of claim 1, wherein the excitable tissue is an area of an infarction in a heart and the alteration of a physiological function is a physiologic change at a cellular level that increases the functionality of cardiac cells selected from the group consisting of an increase of myocardial blood flow through vasodilation, an arrest of arrhythmias, an arrest of aberrant beats and any combination of the foregoing.

12. The method of claim 1, wherein the excitable tissue is an area of an infarction in a heart and the alteration of a physiological function is an alteration leading to a reduction of ischemic tissues that might otherwise undergo necrosis selected from the group consisting of increased bioelectrical activity, improved tissue metabolism, increased blood flow and any combination of the foregoing.

13. The method of claim 1, wherein the alteration of a physiological function is selected from the group consisting of an initiation of an action event, a neural subthreshold bioelectric change, a change in spontaneous rate of cell bioelectrical firing, an alteration of muscle contraction and any combination of the foregoing.

14. A neuromodulation treatment method for a patient, comprising providing at least one microwave source capable of producing a first microwave energy beam with a first electric field and a second microwave energy beam with a second electric field, directing the first microwave energy beam towards a nerve bundle within the patient and directing the second microwave energy beam towards the nerve bundle at an angle from the first microwave energy, wherein the first electric field overlaps with the second electric field at the nerve bundle as to provide a desired neurostimulation effect from the nerve bundle.

15. The method of claim 14, wherein the nerve bundle include an A class of fibers, a B class of fibers and a C class of fibers, further comprising tuning the first microwave energy beam and the second microwave energy beam to direct the desired neurostimulation effect to less than all of the class of fibers.

16. The method of claim 15, wherein the tuning step includes tuning the first microwave energy beam and the second microwave energy beam to direct the desired neurostimulation effect to only one of the class of fibers.

17. The method of claim 14, wherein the tuning step includes tuning a microwave carrier frequency and a pulse modulation frequency for each of the first microwave energy beam and the second microwave energy beam.

18. The method of claim 14, wherein the nerve bundle is in a vertebral region of a back of the patient and the surrounding spinal ganglia regions and the neurostimulation effect is a reduction of pain.

19. A method for treating a patient having skin and deep tissue spaced below the skin by intervening tissue, comprising providing at least one microwave source capable of producing first microwave energy with a first vector electric field, second microwave energy with a second vector electric field and third microwave energy with a third vector electric field, directing each of the first microwave energy, the second microwave energy and the third microwave energy towards the deep tissue, the directions of the first microwave energy, the second microwave energy and the third microwave energy being symmetrically disposed relative to the deep tissue, wherein the first vector electric field, the second vector electric field and the third vector electric field overlap at only the deep tissue so as to treat only the deep tissue.

20. The method of claim 19, wherein the directions of the first microwave energy, the second microwave energy and the third microwave energy are symmetrically disposed in a plane that contains the deep tissue.

21. The method of claim 19, wherein the at least one microwave source includes a microwave antenna spaced from the skin of the patient for each of the first microwave energy, the second microwave energy and the third microwave energy.
